(19) United States
(12) Patent Application Publication
(10) Pub. No.: US 2014/0364869 A1
(43) Pub. Date: Dec. 11, 2014

(54) SYSTEMS AND METHODS FOR STEERING ELECTRICAL STIMULATION LEADS WHILE COUPLED TO A PULSE GENERATOR

(71) Applicant: Boston Scientific Neuromodulation Corporation, Valencia, CA (US)

(72) Inventor: Emarit A. S. Ranu, Fort Collins, CO (US)

(21) Appl. No.: 14/294,084

(22) Filed: Jun. 2, 2014

Related U.S. Application Data
(60) Provisional application No. 61/831,398, filed on Jun. 5, 2013.

(51) Int. Cl. A61N 1/375

(52) U.S. Cl. CPC A61N 1/3752
USPC 606/129; 607/116

(57) ABSTRACT

A control module for an electrical stimulation system includes a connector assembly coupled to a sealed housing. A lead-assembly port is defined along a first end of the connector assembly and extends into the connector assembly. The lead-assembly port is configured for receiving a lead assembly. Connector contacts are disposed in the lead-assembly port and are electrically-coupled to an electronic subassembly disposed in the sealed housing. The connector contacts are configured to electrically-couple to terminals of the lead assembly when the lead assembly is received by the lead-assembly port. A stylet port is defined along a second end of the connector assembly and extends into the interior of the connector assembly. The stylet port opens to the lead-assembly port within the interior of the connector assembly to form a continuous passageway with the lead-assembly port.

### CROSS-REFERENCE TO RELATED APPLICATIONS

This application claims the benefit under 35 U.S.C. §119(e) of U.S. Provisional Patent Application Ser. No. 61/831,398, filed Jun. 5, 2013, which is incorporated herein by reference.

### FIELD

The present invention is directed to the area of implantable electrical stimulation systems and methods of making and using the systems. The present invention is also directed to implantable electrical stimulation systems having control modules that enable insertion of stylets into leads while the leads are attached to the control module, as well as methods of making and using the leads, control modules, and electrical stimulation systems.

### BACKGROUND

Implantable electrical stimulation systems have proven therapeutic in a variety of diseases and disorders. For example, spinal cord stimulation systems have been used as a therapeutic modality for the treatment of chronic pain syndromes. Peripheral nerve stimulation has been used to treat chronic pain syndrome and incontinence, with a number of other applications under investigation. Functional electrical stimulation systems have been applied to restore some functionality to paralyzed extremities in spinal cord injury patients.

Stimulators have been developed to provide therapy for a variety of treatments. A stimulator can include a control module (with a pulse generator), one or more leads, and an array of stimulator electrodes on each lead. The stimulator electrodes are in contact with or near the nerves, muscles, or other tissue to be stimulated. The pulse generator in the control module generates electrical pulses that are delivered by the electrodes to body tissue.

### BRIEF SUMMARY

In one embodiment, a control module for an electrical stimulation system includes a sealed housing having an interior and an exterior. An electronic subassembly is disposed in the interior of the sealed housing. A connector assembly is coupled to the exterior of the housing. The connector assembly has an interior, an outer surface, a first end, and an opposing second end. A lead-assembly port is defined in the outer surface of the first end of the connector assembly and extends into the interior of the connector assembly. The lead-assembly port is configured and arranged for receiving a lead assembly. A plurality of connector contacts is disposed in the lead-assembly port and is electrically coupled to the electronic subassembly. The plurality of connector contacts is configured and arranged to electrically couple to terminals of the lead assembly when the lead assembly is received by the lead-assembly port. A stylet port is defined in the outer surface of the second end of the connector assembly and extends into the interior of the connector assembly. The stylet port opens to the lead-assembly port within the interior of the connector assembly to form a continuous passageway with the lead-assembly port.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of one embodiment of an electrical stimulation system that includes a paddle lead electrically coupled to a control module, according to the invention;

FIG. 2 is a schematic view of one embodiment of an electrical stimulation system that includes a percutaneous lead electrically coupled to a control module, according to the invention;

FIG. 3A is a schematic view of one embodiment of the control module of FIG. 1 and two elongated members of a lead assembly, the control module defining two lead-assembly ports configured for receiving the two elongated members of the lead assembly, the control module, according to the invention;

FIG. 3B is a schematic view of another embodiment of the control module of FIG. 1, the control module defining four lead-assembly ports configured for receiving up to four elongated members of one or more lead assemblies, the control module, according to the invention;

FIG. 3C is a schematic view of one embodiment of an elongated member of the lead assembly of FIG. 3A and a lead extension coupled to the control module of FIG. 1, the lead extension configured to receive the elongated member, according to the invention;

FIG. 4A is a schematic transverse cross-sectional view of one embodiment of an elongated member of the lead assembly of FIG. 3A, the elongated member defining a stylet lumen and multiple conductor lumens, according to the invention;

FIG. 4B is a schematic transverse cross-sectional view of one embodiment of the elongated member of FIG. 4A with conductors extending along conductor lumens defined along the elongated member, according to the invention;

FIG. 5 is a schematic side view of one embodiment of a control module with a lead-assembly port and a stylet port extending from opposing ends of a connector assembly of the control module, the lead-assembly port and the stylet port forming a continuous passageway across the connector assembly; according to the invention;

FIG. 6 is a schematic side view of one embodiment a portion of an elongated member of a lead assembly disposed in a lead-assembly port of the control module of FIG. 5, and a portion of a stylet disposed in a stylet port of the control module, the stylet extending through the stylet port and into a stylet lumen defined in the elongated member, according to the invention; and

FIG. 7 is a schematic overview of one embodiment of components of a stimulation system, including an electronic subassembly disposed within a control module, according to the invention.

### DETAILED DESCRIPTION

The present invention is directed to the area of implantable electrical stimulation systems and methods of making and using the systems. The present invention is also directed to implantable electrical stimulation systems having control modules that enable insertion of stylets into leads while the leads are attached to the control module, as well as methods of making and using the leads, control modules, and electrical stimulation systems.

Suitable implantable electrical stimulation systems include, but are not limited to, a least one lead with one or more electrodes disposed along a distal end of the lead and one or more terminals disposed along the one or more proximal ends of the lead. Leads include, for example, percutaneous leads, paddle leads, and cuff leads. Examples of electrical stimulation systems with leads are found in, for example, U.S. Pat. Nos. 6,181,969; 6,516,227; 6,609,029; 6,609,032; 6,741,892; 7,949,395; 7,244,150; 7,672,734; 7,761,165; 7,974,706; 8,175,710; 8,224,450; and 8,364,278; and U.S. Patent Application Publication No. 2007/0150036, all of which are incorporated by reference.

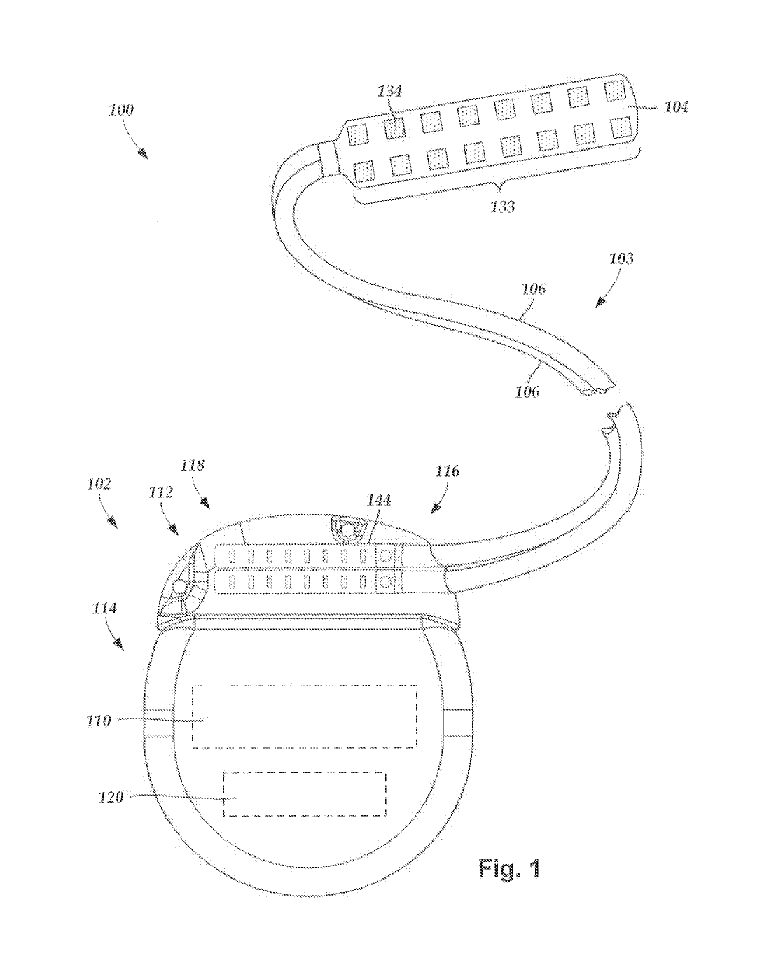

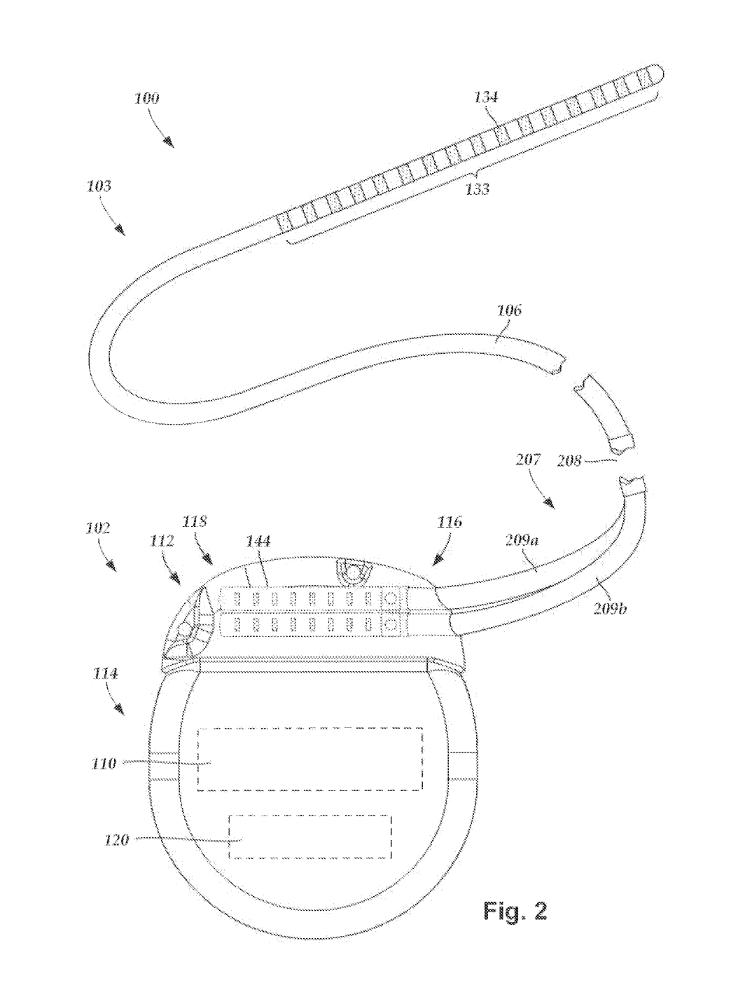

FIG. 1 illustrates schematically one embodiment of an electrical stimulation system 100. The electrical stimulation system includes a control module (e.g., a device that includes a stimulator or pulse generator) 102 and a lead 103 coupleable to the control module 102. The lead 103 includes a paddle body 104 and one or more lead bodies 106. In FIG. 1, the lead 103 is shown having two lead bodies 106. It will be understood that the lead 103 can include any suitable number of lead bodies including, for example, one, two, three, four, five, six, seven, eight or more lead bodies 106. An array of electrodes 133, such as electrode 134, is disposed on the paddle body 104, and an array of terminals (e.g., 210 in FIG. 2A-2B) is disposed along each of the one or more lead bodies 106.

It will be understood that the electrical stimulation system can include more, fewer, or different components and can have a variety of different configurations including those configurations disclosed in the electrical stimulation system references cited herein. For example, instead of a paddle body, the electrodes can be disposed in an array at or near the distal end of a lead body forming a percutaneous lead.

FIG. 2 illustrates schematically another embodiment of the electrical stimulation system 100, where the lead 103 is a percutaneous lead. In FIG. 2, the electrodes 134 are shown disposed along the one or more lead bodies 106. In at least some embodiments, the lead 103 is isodiametric along a longitudinal length of the lead body 106.

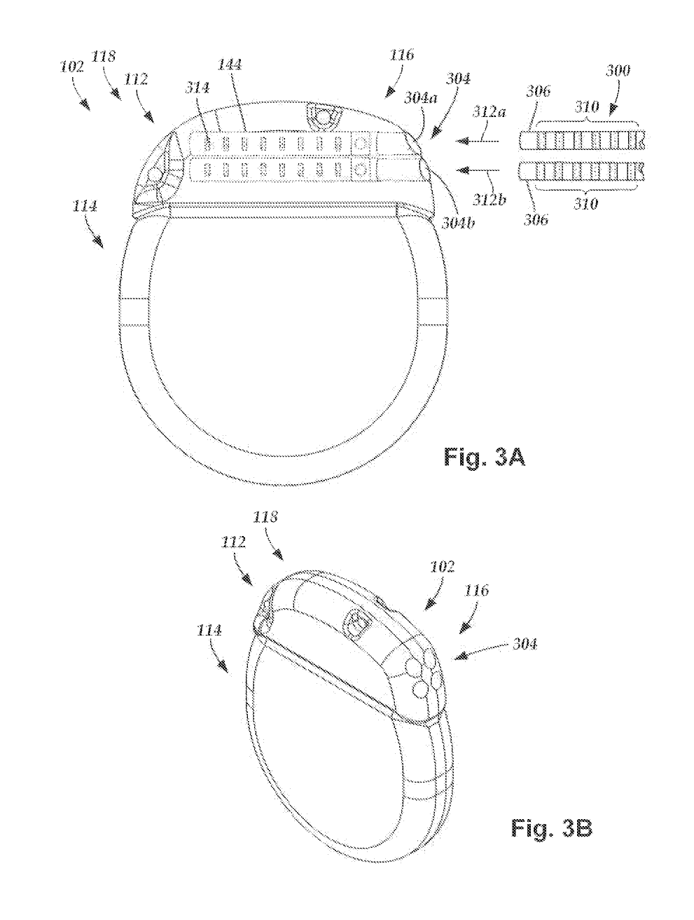

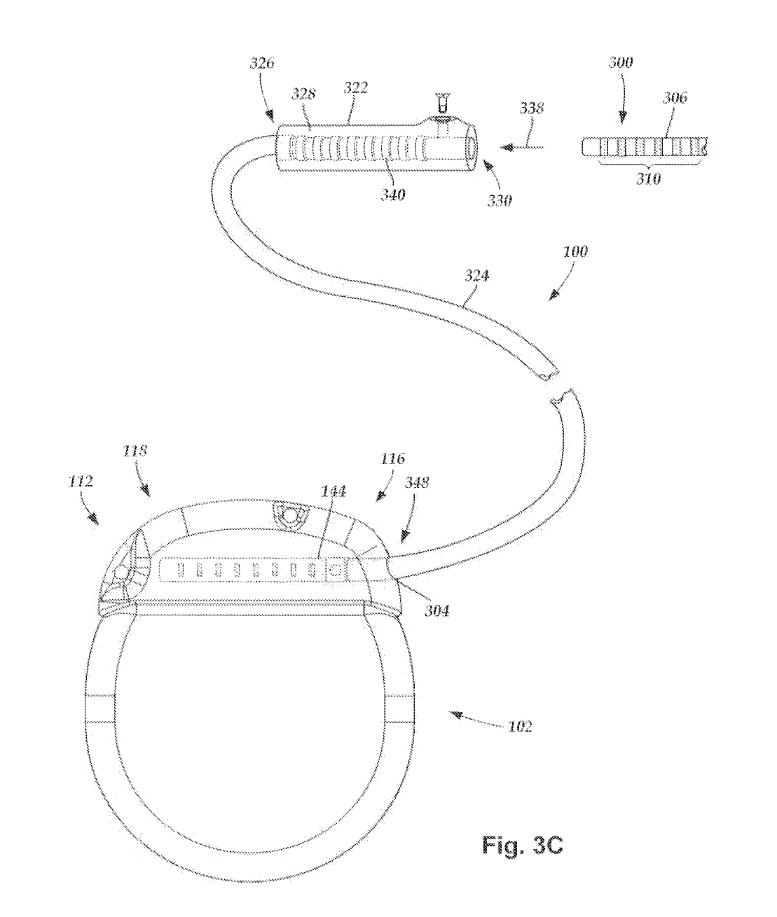

Referring to both FIG. 1 and FIG. 2, the lead 103 can be coupled to the control module 102 in any suitable manner. In at least some embodiments, the lead 103 couples directly to the control module 102. In at least some other embodiments, the lead 103 couples to the control module 102 via one or more intermediate devices (300 in FIGS. 3A-3B). For example, in at least some embodiments one or more lead extensions 324 (see e.g., FIG. 3B) can be disposed between the lead 103 and the control module 102 to extend the distance between the lead 103 and the control module 102. Other intermediate devices may be used in addition to, or in lieu of, one or more lead extensions including, for example, a splitter, an adaptor, or the like or combinations thereof. It will be understood that, in the case where the electrical stimulation system 100 includes multiple elongated devices disposed between the lead 103 and the control module 102, the intermediate devices may be configured into any suitable arrangement.

In FIG. 2, the electrical stimulation system 100 is shown having a splitter 207 configured and arranged for facilitating coupling of the lead 103 to the control module 102. The splitter 207 includes a splitter connector 208 configured to couple to a proximal end of the lead 103, and one or more splitter tails 209a and 209b configured and arranged to couple to the control module 102 (or another splitter, a lead extension, an adaptor, or the like).

Referring to both FIG. 1 and FIG. 2, the control module 102 typically includes a connector housing 112, or “header,” and a sealed electronics housing 114. An electronic subassembly 110 and an optional power source 120 are disposed in the sealed electronics housing 114. The connector housing 112 is disposed along a portion of an exterior surface of the sealed electronics housing 114 and includes a first end 116 and an opposing second end 118.

A control-module connector 144 is disposed in the connector housing 112. The control-module connector 144 is configured and arranged to receive, either directly or indirectly, a portion of the lead 103 and make an electrical connection between the lead 103 and the electronic subassembly 110 of the control module 102.

The electrical stimulation system, or components of the electrical stimulation system, including the paddle body 104, the one or more of the lead bodies 106, and the control module 102, are typically implanted into the body of a patient. The electrical stimulation system can be used for a variety of applications including, but not limited to deep brain stimulation, neural stimulation, spinal cord stimulation, muscle stimulation, and the like.

The electrodes 134 can be formed using any conductive, biocompatible material. Examples of suitable materials include metals, alloys, conductive polymers, conductive carbon, and the like, as well as combinations thereof. In at least some embodiments, one or more of the electrodes 134 are formed from one or more of: platinum, platinum iridium, palladium, palladium rhodium, or titanium.

Any suitable number of electrodes 134 can be disposed on the lead including, for example, four, five, six, seven, eight, nine, ten, eleven, twelve, fourteen, sixteen, twenty-four, thirty-two, or more electrodes 134. In the case of paddle leads, the electrodes 134 can be disposed on the paddle body 104 in any suitable arrangement. In FIG. 1, the electrodes 134 are arranged into two columns, where each column has eight electrodes 134.

The electrodes of the paddle body 104 (or one or more lead bodies 106) are typically disposed in, or separated by, a non-conductive, biocompatible material such as, for example, silicone, polyurethane, polyetheretherketone (“PEEK”), epoxy, and the like or combinations thereof. The one or more lead bodies 106 and, if applicable, the paddle body 104 may be formed in the desired shape by any process including, for example, molding (including injection molding), casting, and the like. The non-conductive material typically extends from the distal ends of the one or more lead bodies 106 to the proximal end of each of the one or more lead bodies 106.

In the case of paddle leads, the non-conductive material typically extends from the paddle body 104 to the proximal end of each of the one or more lead bodies 106. Additionally, the non-conductive, biocompatible material of the paddle body 104 and the one or more lead bodies 106 may be the same or different. Moreover, the paddle body 104 and the one or more lead bodies 106 may be a unitary structure or can be formed as two separate structures that are permanently or detachably coupled together.

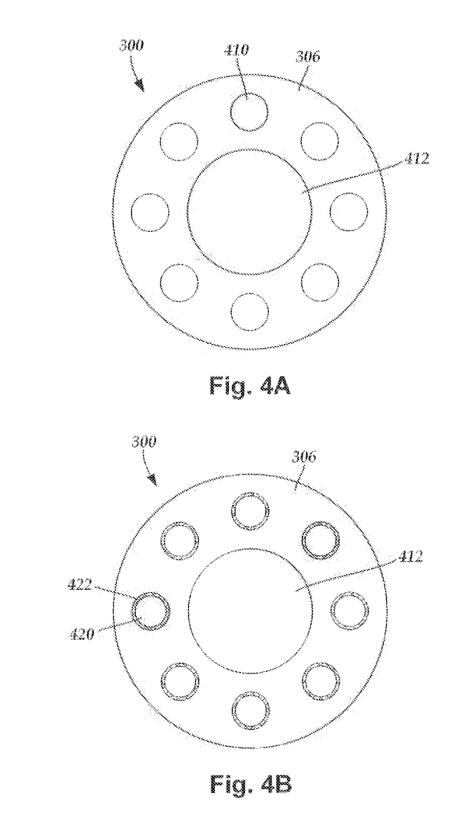

Terminals (e.g., 310 in FIGS. 3A-3B) are typically disposed along the proximal end portion of the one or more lead bodies 106 of the electrical stimulation system 100 (as well as along proximal end portions of any splitters, lead extensions, adaptors, or the like) for electrical connection to corresponding connector contacts (e.g., 314 in FIGS. 3A-3B). The connector contacts are disposed in connectors (e.g., 144 in FIGS. 1-3B; and 322 FIG. 3B) which, in turn, are disposed on, for example, the control module 102 (or along a distal end portion of a lead extension, a splitter, an adaptor, or the like). Electrically-conductive wires, cables, or the like (“conductors”) (see e.g., 420 in FIG. 4B) extend from, in the case of lead bodies, the terminals to the electrodes 134. In the case of intermediate devices (e.g., lead extensions, adaptors, splitters, or the like), the conductors extend from terminals to connector contacts of connectors (see e.g., connector contacts 340 of lead-extension connector 322 of FIG. 3C). Typically, one or more electrodes 134 are electrically coupled to each terminal. In at least some embodiments, each terminal is only connected to one electrode 134.

The conductors may be embedded in the non-conductive material of the lead body 106 (or other elongated members, such as lead extensions, splitters, adaptors, or the like) or can be disposed in one or more lumens (see e.g., 410 of FIG. 4A) extending along the lead body 106 (or other elongated member). In some embodiments, there is an individual lumen for each conductor. In other embodiments, two or more conductors extend through a lumen. There may also be one or more stylet lumens (see e.g., 412 of FIG. 4A) that open at, or near, the proximal end of the one or more lead bodies 106, for example, for inserting a stylet to facilitate placement of the one or more lead bodies 106 within a body of a patient. Additionally, there may be one or more lumens (not shown) that open at, or near, the distal end of the one or more lead bodies 106 (or other elongated members), for example, for infusion of drugs or medication into the site of implantation. In at least one embodiment, the one or more lumens are flushed continually, or on a regular basis, with saline, epidural fluid, or the like. In at least some embodiments, the one or more lumens are permanently or removably sealable along distal ends of the lumens.

FIG. 3A is a schematic side view of one embodiment of proximal end portions of two elongated members 306 of a lead assembly 300 configured and arranged for coupling to one embodiment of the control-module connector 144. The elongated members 306 of the lead assembly 300 may include, for example, one or more of the lead bodies (e.g., the lead bodies 106 of FIG. 1 or FIG. 2), one or more intermediate devices (e.g., the splitter 207, the lead extension 324 of FIG. 3C, an adaptor, or the like or combinations thereof), or a combination thereof.

The control-module connector 144 defines at least one lead-assembly port 304 into which a proximal end portion of the lead assembly 300 can be inserted, as shown by directional arrows 312a and 312b. In FIG. 3A (and in other figures), the connector housing 112 is shown having two lead-assembly ports 304a and 304b. The connector housing 112 can define any suitable number of lead-assembly ports including, for example, one, two, three, four, five, six, seven, eight, or more lead-assembly ports. FIG. 3B illustrates an alternate embodiment of the control module 102 with four lead-assembly ports 304 disposed in the connector housing 112. The lead-assembly ports 304 shown in each of FIGS. 3A-3B extend from the first end 116 of the connector housing 112.

As shown in FIG. 3A, the control-module connector 144 also includes a plurality of connector contacts, such as connector contact 314, disposed within each lead-assembly port 304a and 304b. When the one or more lead assemblies 300 are inserted into the one or more lead-assembly ports 304a and 304b, the connector contacts 314 can be aligned with terminals 310 disposed along the proximal end portion(s) of the one or more lead assemblies 300 to electrically couple the control module 102 to the electrodes (134 of FIGS. 1 or 2). Examples of connectors in control modules are found in, for example, U.S. Pat. Nos. 7,244,150 and 8,224,450, which are incorporated by reference.

FIG. 3C is a schematic side view of another embodiment of the electrical stimulation system 100. The electrical stimulation system 100 includes a lead extension 324 that is configured and arranged to couple one or more elongated members (e.g., one or more lead bodies, splitters, adaptors, another lead extension, or the like or combinations thereof) of the lead assembly 300 to the control module 102. In FIG. 3B, the lead extension 324 is shown coupled to a single lead-assembly port 304 defined in the control-module connector 144. Additionally, the lead extension 324 is shown configured and arranged to couple to a single elongated member 306. In alternate embodiments, the lead extension 324 is configured and arranged to couple to multiple lead-assembly ports 304 defined in the control-module connector 144, or to receive multiple elongated members, or both.

A lead-extension connector 322 is disposed on the lead extension 324. In FIG. 3B, the lead-extension connector 322 is shown disposed along a distal end portion 326 of the lead extension 324. The lead-extension connector 322 includes a connector housing 328. The connector housing 328 defines at least one lead-assembly port 330 into which terminals 310 of the elongated device can be inserted, as shown by directional arrow 338. The connector housing 328 also includes a plurality of connector contacts, such as connector contact 340. When the elongated device 306 is inserted into the lead-assembly port 330, the connector contacts 240 disposed in the connector housing 328 can be aligned with the terminals 310 of the elongated device to electrically couple the lead extension 324 to the electrodes (134 of FIGS. 1 and 2) disposed along the lead (103 in FIGS. 1 and 2).

In at least some embodiments, a proximal end portion 348 of the lead extension 324 is similarly configured and arranged as a proximal end portion of the lead 103 (or other elongated member 306). The lead extension 324 may include a plurality of conductors (see e.g., 420 in FIG. 4B) that electrically couple the connector contacts 340 to the proximal end portion 348 of the lead extension 324 that is opposite to the distal end portion 326. In at least some embodiments, the conductors disposed in the lead extension 324 can be electrically coupled to a plurality of terminals (not shown) disposed along the proximal end portion 348 of the lead extension 324. In at least some embodiments, the proximal end portion 348 of the lead extension 324 is configured and arranged for insertion into a connector disposed in another lead extension (or another intermediate device). In other embodiments (and as shown in FIG. 3B), the proximal end portion 348 of the lead extension 324 is configured and arranged for insertion into the control-module connector 144.

Turning to FIGS. 4A-4B, as mentioned above the proximal end portion(s) of one or more elongated members 306 of the lead assembly 300 are configured for insertion into the lead-assembly port(s) of the connector assembly of the control module. The one or more lead assemblies may include, for example, one or more of the lead bodies, one or more intermediate devices (e.g., a splitter, a lead extension, an adaptor, or the like or combinations thereof), or a combination thereof.

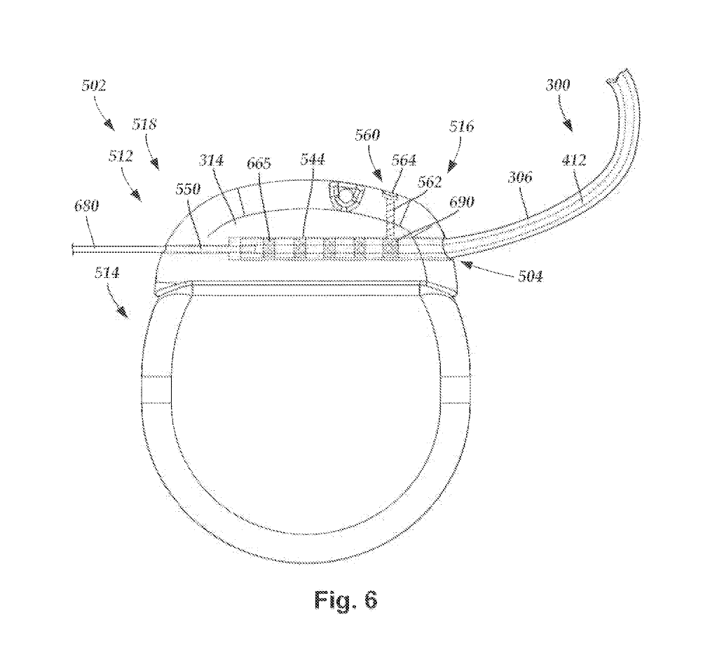

FIGS. 4A-4B illustrate, in transverse cross-section, the elongated member 306 of the lead assembly 300. Conductor lumens, such as conductor lumen 410, are defined in the elongated member 306. Also defined in the elongated member 306 is a stylet lumen 412 configured and arranged to receive a stylet (see e.g., 680 in FIG. 6). FIG. 4B additionally shows conductors, such as conductor 420, disposed in the conductor lumens 410. The conductors 420 may, optionally, be encased in electrically-nonconductive material to maintain electrical isolation of the conductors 420. The elongated member 306 shown in FIGS. 4A-4B may be any suitable structure of the lead assembly 300 suitable for coupling with the control-module connector including, for example, a lead body, a splitter, a lead extension, an adaptor, or the like.

Typically, each of the one or more elongated members of the lead assembly include terminals disposed along a proximal end portion of the elongated member and conductors extending along a longitudinal length of the elongated member from the terminals to connector contacts of a connector (see e.g., 322 in FIG. 3C) disposed along a distal end portion of the elongated device (in the case of an intermediate device) or to electrodes (see e.g., 134 in FIGS. 1 and 2) (in the case of a lead body).

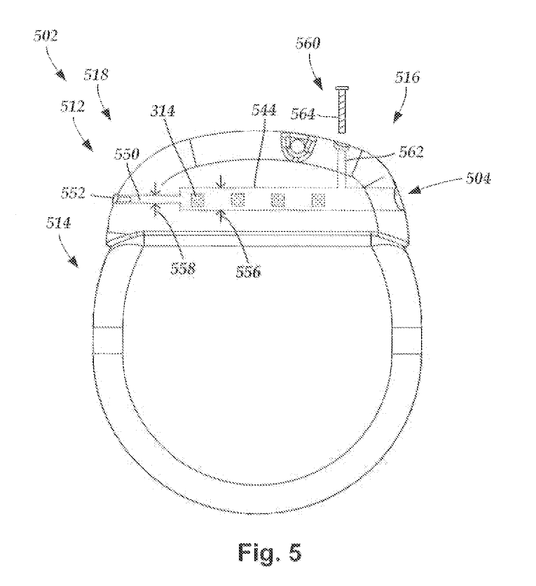

Turning to FIGS. 5-6, over time an implanted lead assembly may lose the ability to provide efficacious treatment to a patient at the current stimulation location. For example, the lead assembly may migrate away from a previous target stimulation location, or a disease state may change, or the like. In which case, the electrodes may need to be moved to a new stimulation location to regain lost efficacy. Unfortunately, moving the electrodes while the lead assembly is implanted in the patient may involve performing a “revision” surgery.

A revision surgery may involve making an incision into the patient to access the implanted lead assembly in order to move the electrodes to a new stimulation location. Leads (and intermediate devices) however, are typically floppy and not easily moved without the aid of a stiffening device (e.g., a stylet, or the like) to increase the rigidity of the lead.

In at least some instances, a revision surgery may involve inserting a stylet into stylet lumens extending along the longitudinal length of one or more elongated members of the lead assembly. Stylet lumens are typically accessible from the proximal end of the lead assembly, where the lead assembly couples to the control module. Once the stylet lumen is accessed and the stylet is advanced along the stylet lumen, the distal end of the lead can be moved to position the electrodes in proximity to the new stimulation location. Thereafter, the stylet may be removed and the incision closed.

Typically, the proximal end of the lead assembly is uncoupled from the control module in order to gain access to the stylet lumen. In which case, guiding of the distal end of the lead is performed while the proximal end of the lead assembly is uncoupled from the control module. Once the distal end of the lead is guided to the new stimulation location and the stylet is removed from the stylet lumen, the proximal end of the lead assembly is typically re-coupled to the control module in order to re-enable stimulation.

Uncoupling and re-coupling the lead assembly to the control module may introduce wear on the system parts and may cause electrical and/or mechanical failure. Moreover, reconnecting the lead assembly sometimes may result in misalignment of the terminals coupleable with the connector contacts of the control module. Additionally, when the lead assembly is temporarily removed from the control-module connector, there is a potential for patient tissue or other surgical materials to be undesirably deposited on the lead assembly and cause a corresponding increase in electrical impedance, thereby potentially reducing therapeutic efficiency. Furthermore, uncoupling and re-coupling of the lead assembly to the control module is time-consuming and laborious for the medical team and may add additional exposure to risk and post-operative discomfort for the patient.

As herein described, a control module is disclosed that enables the distal end of the lead to be guided during a revision surgery while being simultaneously coupled to the control module. Thus, the disclosed control module enables the distal end of the lead to be guided within the patient without first needing to uncouple the proximal end of the lead assembly from the control module. Additionally, the disclosed control module enables the distal end of the lead to be guided within the patient without needing to re-couple the proximal end of the lead assembly to the control module after the distal end of the lead has been moved to the new stimulation location.

FIG. 5 schematically illustrates one embodiment of a control module 502 for an electrical stimulation system. The control module 502 includes an electronics housing 514 having an interior, an outer surface, and a connector housing 512 disposed along a portion of the outer surface of the electronics housing 514. An electronic subassembly (110 in FIG. 1) is disposed in the interior of the electronics housing 514.

The connector housing 512 includes an interior, an outer surface, a first end 516, and an opposing second end 518. A control-module connector 544 is disposed in the connector housing 512. The control-module connector 544 defines a lead-assembly port 504 for receiving a proximal end portion of the elongated member of the lead assembly (e.g., the proximal end portion of the lead or other intervening device, such as a lead extension, adaptor, splitter, or the like). In at least some embodiments, the lead-assembly port 504 is defined along the outer surface of the first end 516 of the connector housing 512 and extends from the first end 516 of the connector housing 512 into the interior of the connector housing 512.

Connector contacts 314 are disposed within the lead-assembly port 504 and are electrically coupled to the electronic subassembly disposed in the interior of the electronics housing 514. In at least some embodiments, the connector contacts 314 are arranged along the lead-assembly port 504 such that terminals disposed along the proximal end portion of the elongated member of the lead assembly align with, and electrically couple to, the connector contacts when the proximal end portion of the elongated member of the lead assembly is inserted into the lead-assembly port.

The control-module connector 544 can be formed from any suitable biocompatible material including, for example, silicone, polyurethane, and the like or combinations thereof. The connector contacts 314 can be formed from any suitable conductive, biocompatible material including, for example, metals, alloys, conductive polymers, and the like or combinations thereof. In FIG. 5, four connector contacts 314 are shown disposed in the control-module connector 544. It will be understood that there can be any suitable number of connector contacts disposed in the control-module connector 544 including, for example, one, two, three, four, five, six, seven, eight, nine, ten, eleven, twelve, thirteen, fourteen, fifteen, sixteen, twenty, twenty-four, twenty-eight, thirty-two, or more connector contacts 314. As will be recognized, other numbers of connector contacts 314 are also possible.

Optionally, the control module 560 includes a retention assembly 560 for removably fastening the proximal end portion of the elongated member of the lead assembly in the lead-assembly port 504 during operation of the electrical stimulation system. In at least some embodiments, the retention assembly 560 includes a fastener aperture 562 and a fastener 564 for inserting into the fastener aperture 562 and for tightening against the proximal end portion of the elongated member of the lead assembly when inserted into the lead-assembly port 504.

In FIG. 5, the fastener aperture 562 is shown extending along the interior of the connector housing 512 between the outer surface of the connector housing 512 and the lead-assembly port 504. In at least some embodiments, the fastener aperture 562 extends in a direction that is perpendicular, or approximately perpendicular, to the lead-assembly port 504. In FIG. 5, the fastener 564 is shown as a set screw. Other types of fasteners, however, may be used as well, such as pins, posts, and the like or combinations thereof. Alternately or additionally, the retention assembly may include a press fit mechanism, interference fit or the like.

A stylet port 550 is defined along the outer surface of the second end 518 of the connector housing 512 and extends into the interior of the connector housing 512. The stylet port 550 is sized to receive a stylet (680 in FIG. 6). The stylet port 550 opens to the lead-assembly port 504 within the interior of the connector housing 512 such that the stylet port 550 and the lead-assembly port 504 collectively form a continuous passageway extending across the entire connector housing 512 from the first end 516 of the connector housing 512 to the second end 518 of the connector housing 514. In at least some embodiments, the stylet port 550 and the lead-assembly port 504 extend parallel to one another. In at least some embodiments, the stylet port 550 and the lead-assembly port 504 collectively form a continuous linear passageway extending across the connector housing 512.

It may be advantageous to prevent fluid from entering the connector housing 512, via the stylet port 550, during operation of the control module 502. A removable plug 552 may, optionally, be insertable into the stylet port 550 to reduce, or even prevent, fluid, or other undesired materials, from entering the interior of the connector housing 512 through the stylet port 550. In at least some embodiment, the removable plug 552 also prevents fluid, or (or other undesired materials, from entering the proximal end portion of the elongated member of the lead assembly when the proximal end portion of the elongated member of the lead assembly is received by the lead-assembly port 504.

The removable plug 552 can be located at any suitable location on or around the stylet lumen 550. For example, the removable plug 552 can be disposed along the outer surface of the connector housing 512 over an opening of the lead-assembly port 504, or the removable plug 552 can be disposed at least partially within the stylet port 550, or both. Optionally, the plug 552 may be tethered to the connector housing to prevent loss of the plug 552 when the plug 552 is removed to provide access to the stylet port 504. The plug 552 can couple with the stylet port 550 in any suitable manner including, for example, an interference fit, interconnecting threads, or the like or combinations thereof.

The lead-assembly port 504 has a diameter 556 suitable for receiving the proximal end portion of the elongated member of the lead assembly. The stylet port 550 has a diameter 558 suitable for receiving a stylet. In at least some embodiments, the diameter 556 of the lead-assembly port 504 is smaller than the diameter 558 of the stylet port 550. In at least some embodiments, the diameter 556 of the lead-assembly port 504 is larger than the diameter 558 of the stylet port 550. The diameter 556 of the lead-assembly port 504 can be, for example, 10%, 20%, 30%, 40%, 50%, 60%, 70%, 80%, 90%, 100%, 150%, 200% or more larger than the diameter 558 of the stylet port 550. In some embodiments, the diameter 556 of the lead-assembly port 504 is at least 20% larger and no greater than 150% larger than the diameter 558 of the stylet port 550. In other embodiments, the diameter 556 of the lead-assembly port 504 is at least 50% larger and no greater than 100% larger than the diameter 558 of the stylet port 550.

In FIG. 5, the control module 502 is shown having a single stylet port, and a single lead-assembly port. The control module 502 can have any suitable number of lead-assembly ports 504 including, for example, two, three, four, five, six, seven, eight, or more lead-assembly ports 504. The control module 502 can have any suitable number of stylet ports 550 including, for example, two, three, four, five, six, seven, eight, or more stylet ports 550. In some embodiments, the number of stylet ports 550 is equal to the number of lead-assembly ports 504. In other embodiments, the number of stylet ports 550 is either less than or greater than to the number of lead-assembly ports 504.

Turning to FIG. 6, the stylet port 550 is configured and arranged for receiving a stylet while the proximal end portion of the elongated member of the lead assembly is received by the lead-assembly port 504. In at least some embodiments, the stylet can be inserted into the stylet port 550 and advanced into the stylet lumen (412 in FIG. 4) of the lead assembly and used to provide enough stiffness to the lead assembly to enable movement of the distal end portion of the lead to a new stimulation location.

FIG. 6 schematically illustrates one embodiment the control module 502 with a proximal end portion of the elongated member 306 of the lead assembly 300 disposed in the lead-assembly port 504 and a portion of a stylet 680 disposed in the stylet port 550. In FIG. 6, the stylet 680 is shown extending through the stylet port 550 and partially into the stylet lumen 412 of the elongated member 306 while the proximal end portion of the elongated member 306 is disposed in the lead-assembly port 504.

The stylet 680 is configured for providing rigidity to the lead assembly to facilitate advancement of the lead assembly when implanted into the patient. The stylet 680 can be formed from any suitable biocompatible material with enough rigidity for stiffening a lead assembly enough to be guided within the patient while also being flexible enough to negotiate anatomical twists and turns. Examples of suitable materials include, for example, tungsten, stainless steel, plastic, or the like or combinations thereof. The stylet 680 may, optionally, have a handle to facilitate gripping of the stylet 680 during use.

The proximal end of the elongated member of the lead assembly may be firmly secured within the control-module connector 544 during movement of the lead assembly with the aid of the retention assembly 560. In at least some embodiments, a retention sleeve 690 is disposed around at least a portion of the body of the elongated member 306. The retention sleeve 690 may provides a rigid surface against which the fastener 564 of the retention assembly 560 can be tightened against when the fastener 564 is inserted into the fastener aperture 562. The retention sleeve 690 can be formed using any rigid, biocompatible material including, for example, metals, alloys, rigid polymers, rigid carbon, and the like, as well as combinations thereof.

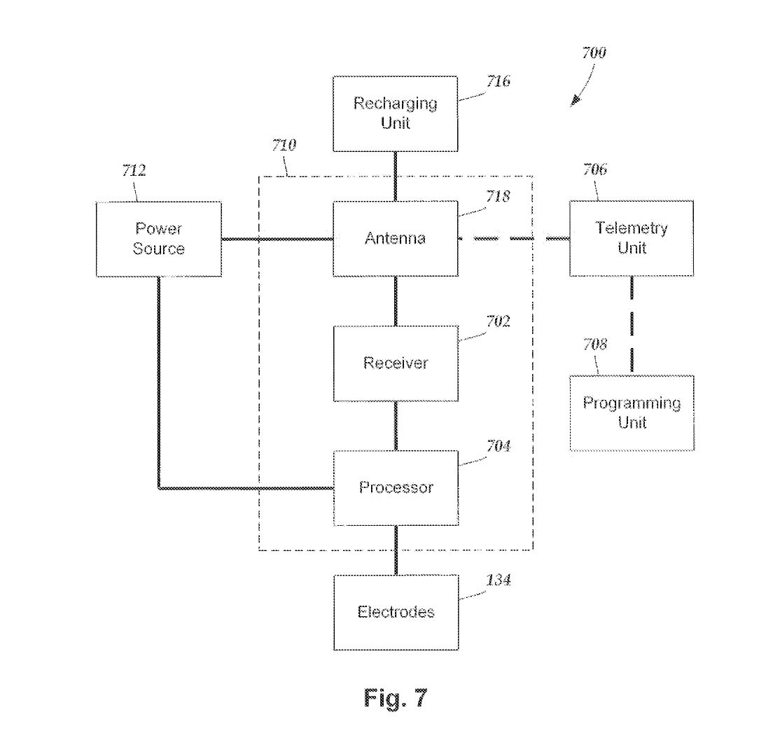

FIG. 7 is a schematic overview of one embodiment of components of an electrical stimulation system 700 including an electronic subassembly 710 disposed within a control module. It will be understood that the electrical stimulation system can include more, fewer, or different components and can have a variety of different configurations including those configurations disclosed in the stimulator references cited herein.

Some of the components (for example, a power source 712, an antenna 718, a receiver 702, and a processor 704) of the electrical stimulation system can be positioned on one or more circuit boards or similar carriers within a sealed housing of an implantable pulse generator, if desired. Any power source 712 can be used including, for example, a battery such as a primary battery or a rechargeable battery. Examples of other power sources include super capacitors, nuclear or atomic batteries, mechanical resonators, infrared collectors, thermally-powered energy sources, flexural powered energy sources, bioenergy power sources, fuel cells, bioelectric cells, osmotic pressure pumps, and the like including the power sources described in U.S. Pat. No. 7,437,193, incorporated herein by reference.

As another alternative, power can be supplied by an external power source through inductive coupling via the optional antenna 718 or a secondary antenna. The external power source can be in a device that is mounted on the skin of the user or in a unit that is provided near the user on a permanent or periodic basis.

If the power source 712 is a rechargeable battery, the battery may be recharged using the optional antenna 718, if desired. Power can be provided to the battery for recharging by inductively coupling the battery through the antenna to a recharging unit 716 external to the user. Examples of such arrangements can be found in the references identified above.

In one embodiment, electrical current is emitted by the electrodes 134 on the paddle or lead body to stimulate nerve fibers, muscle fibers, or other body tissues near the electrical stimulation system. The processor 704 is generally included to control the timing and electrical characteristics of the electrical stimulation system. For example, the processor 704 can, if desired, control one or more of the timing, frequency, strength, duration, and waveform of the pulses. In addition, the processor 704 can select which electrodes can be used to provide stimulation, if desired. In some embodiments, the processor 704 selects which electrode(s) are cathodes and which electrode(s) are anodes. In some embodiments, the processor 704 is used to identify which electrodes provide the most useful stimulation of the desired tissue.

Any processor can be used and can be as simple as an electronic device that, for example, produces pulses at a regular interval or the processor can be capable of receiving and interpreting instructions from an external programming unit 708 that, for example, allows modification of pulse characteristics. In the illustrated embodiment, the processor 704 is coupled to a receiver 702 which, in turn, is coupled to the optional antenna 718. This allows the processor 704 to receive instructions from an external source to, for example, direct the pulse characteristics and the selection of electrodes, if desired.

In one embodiment, the antenna 718 is capable of receiving signals (e.g., RF signals) from an external telemetry unit 706 which is programmed by the programming unit 708. The programming unit 708 can be external to, or part of, the telemetry unit 706. The telemetry unit 706 can be a device that is worn on the skin of the user or can be carried by the user and can have a form similar to a pager, cellular phone, or remote control, if desired. As another alternative, the telemetry unit 706 may not be worn or carried by the user but may only be available at a home station or at a clinician's office. The programming unit 708 can be any unit that can provide information to the telemetry unit 706 for transmission to the electrical stimulation system 700. The programming unit 708 can be part of the telemetry unit 706 or can provide signals or information to the telemetry unit 706 via a wireless or wired connection. One example of a suitable programming unit is a computer operated by the user or clinician to send signals to the telemetry unit 706.

The signals sent to the processor 704 via the antenna 718 and the receiver 702 can be used to modify or otherwise direct the operation of the electrical stimulation system. For example, the signals may be used to modify the pulses of the electrical stimulation system such as modifying one or more of pulse duration, pulse frequency, pulse waveform, and pulse strength. The signals may also direct the electrical stimulation system 700 to cease operation, to start operation, to start charging the battery, or to stop charging the battery. In other embodiments, the stimulahon system does not include the antenna 718 or receiver 702 and the processor 704 operates as programmed.

Optionally, the electrical stimulation system 700 may include a transmitter (not shown) coupled to the processor 704 and the antenna 718 for transmitting signals back to the telemetry unit 706 or another unit capable of receiving the signals. For example, the electrical stimulation system 700 may transmit signals indicating whether the electrical stimulation system 700 is operating properly or not or indicating when the battery needs to be charged or the level of charge remaining in the battery. The processor 704 may also be capable of transmitting information about the pulse characteristics so that a user or clinician can determine or verify the characteristics.

The above specification, examples and data provide a description of the manufacture and use of the composition of the invention. Since many embodiments of the invention can be made without departing from the spirit and scope of the invention, the invention also resides in the claims hereinafter appended.

What is claimed as new and desired to be protected by Letters Patent of the United States is:

1. A control module for an electrical stimulation system, the control module comprising:
a sealed housing having an interior and an exterior;
an electronic subassembly disposed in the interior of the sealed housing;
a connector assembly coupled to the exterior of the sealed housing, the connector assembly having an interior, an outer surface, a first end, and an opposing second end;
a lead-assembly port defined in the outer surface of the first end of the connector assembly and extending into the interior of the connector assembly, the lead-assembly port configured and arranged for receiving a lead assembly;
a plurality of connector contacts disposed in the lead-assembly port and electrically coupled to the electronic subassembly, the plurality of connector contacts configured and arranged to electrically couple to terminals of the lead assembly when the lead assembly is received by the lead-assembly port; and
a stylet port defined in the outer surface of the second end of the connect assembly and extending into the interior of the connector assembly;
wherein the stylet port opens to the lead-assembly port within the interior of the connector assembly to form a continuous passageway with the lead-assembly port.

2. The control module of claim 1, wherein the stylet port is configured and arranged for receiving a stylet while the lead assembly is received by the lead-assembly port.

3. The control module of claim 1, wherein the continuous passageway formed by the lead-assembly port and the stylet port extends linearly across the connector assembly from the first end to the second end.

4. The control module of claim 1, wherein the lead-assembly port has a diameter and the stylet port has a diameter, and wherein the diameter of the lead-assembly port is larger than the diameter of the stylet port.

5. The control module of claim 4, wherein the diameter of the lead-assembly port is at least 10% larger than the diameter of the stylet port.

6. The control module of claim 1, further comprising a removable plug insertable into the stylet port, the removable plug configured and arranged to prevent fluid from entering the interior of the connector assembly through the stylet port when the removable plug is installed in the stylet port.

7. The control module of claim 1, further comprising a retention assembly defining an aperture extending along the interior of the connector assembly between the outer surface of the connector assembly and the lead-assembly port.

8. The control module of claim 1, wherein the lead-assembly port is a first lead-assembly port and the stylet port is a first stylet port, and wherein the connector assembly further defines
a second lead-assembly port defined in the outer surface of the first end of the connector assembly and extending into the interior of the connector assembly; and
a second stylet port defined in the outer surface of the second end of the connector assembly and extending into the interior of the connector assembly;
wherein the second stylet port opens to the second lead-assembly port within the interior of the connector assembly to form a continuous passageway with the second lead-assembly port.

9. An electrical stimulation system comprising:
a lead assembly comprising
at least one lead body having a distal end portion, a proximal end portion, and a longitudinal length,
a plurality of electrodes disposed along the distal end portion of the at least one lead body,
a plurality of terminals disposed along the proximal end portion of the at least one lead body, and
a plurality of conductors, each conductor of the plurality of conductors electrically coupling each of the plurality of terminals to at least one of the plurality of electrodes; and the control module of claim 1 coupleable to the lead assembly.

10. The electrical stimulation system of claim 9, further comprising a stylet configured and arranged for insertion into the stylet port of the control module while the lead assembly is inserted into the lead-assembly port.

11. The electrical stimulation system of claim 10, wherein the lead assembly defines a stylet lumen extending along the lead assembly, the stylet lumen configured and arranged to receive the stylet when the stylet is inserted into the stylet port and the lead assembly is received by the lead-assembly port.

12. The electrical stimulation system of claim 9, wherein the lead assembly comprises a retention sleeve.

13. The electrical stimulation system of claim 12, wherein the control module further comprises a retention assembly, the retention assembly defining an aperture extending along the interior of the connector assembly between the outer surface of the connector assembly and the lead-assembly port.

14. The electrical stimulation system of claim 13, wherein the aperture of the retention assembly is configured and arranged to receive a fastener coupleable to the retention sleeve of the lead assembly when the lead assembly is inserted into the lead-assembly port.

15. The electrical stimulation system of claim 9, wherein the lead assembly comprises a percutaneous lead.

16. The electrical stimulation system of claim 9, wherein the lead assembly comprises a paddle lead.

17. The electrical stimulation system of claim 9, wherein the lead assembly comprises a lead extension configured and arranged to couple the at least one lead body to the control module.

18. A method of steering an electrical stimulation lead, the method comprising:
advancing a lead assembly into a patient, the lead assembly comprising a plurality of electrodes disposed along a distal end portion of the lead assembly;
providing the control module of claim 1;
inserting a proximal end portion of the lead assembly into the lead-assembly port of the control module;
inserting a stylet into the stylet port of the control module while the proximal end portion of the lead assembly is inserted into the lead-assembly port;
advancing the stylet into a stylet lumen defined along the lead assembly; and
using the stylet to steer the distal end portion of the lead assembly within the patient.

19. The method of claim 18, further comprising removing the stylet from the stylet lumen and the stylet port after using the stylet to steer the distal end portion of the lead assembly within the patient.

20. The method of claim 19, further comprising sealing the stylet port after removing the stylet from the stylet lumen to prevent fluid from entering the connector assembly of the control module through the stylet port.
